(19) United States
(12) Patent Application Publication
(10) Pub. No.: US 2005/0148042 A1
(43) Pub. Date: Jul. 7, 2005

(54) Hybrid phosphoinositide phospholipids: compositions and uses

(76) Inventors: Glenn Prestwich, Salt Lake City, UT (US); Piotr W. Rzepecki, Salt Lake City, UT (US); Colin G. Ferguson, Salt Lake City, UT (US); Paul O. Neilsen, West Valley City, UT (US); Angie M. Branch, South Jordan, UT (US); Lee R. Crosby, Hyrum, UT (US)

Correspondence Address: THE MCCALLUM LAW FIRM, LLC, 132 KOLAR COURT, ERIE, CO, 80516, US

(21) Appl. No.: 10/510,197

(22) PCT Filed: Mar. 31, 2003
(86) PCT No.: PCT/US03/09858

Related U.S. Application Data
(60) Provisional application No. 60/368,556, filed on Mar. 29, 2002.
(60) Provisional application No. 60/392,783, filed on Jun. 28, 2002.

(51) Int. Cl. C12Q 1/48; C07F 9/655; C07F 9/144; C07K 14/47

(52) U.S. Cl. 435/15; 548/113; 530/352; 558/156; 549/218

(57) ABSTRACT

The methods and compositions disclosed herein concern the synthesis of a novel class of “two-headed” phospholipid-phosphoinositide hybrids possessing a carbon backbone, such as 2,3-diacylthreitol, erythritol or a synthetic module. The second phospholipid head group allows introduction of a biochemical or chemical moiety in a position orthogonal in space to those occupied by the phosphoinositide head group and the two acyl chains. The diacyl moieties allow for the incorporation of Pea-PIP₂ into a lipid bilayer, while the Ptdlns(4,5)P₂ moiety in the aqueous layer is specifically recognized by lipid binding proteins. In alternative embodiments of the invention, reporters, for example biotin, fluorophores and/or spin labels, are attached to the free amino group of the head groups of such molecules to specifically target the reporters to the lipid-water interface.

### CROSS REFERENCE TO RELATED APPLICATIONS

This application claims priority from U.S. Provisional Patent Application No. 60/368,556 filed Mar. 29, 2002 and 60/392,783 filed Jun. 28, 2002.

### STATEMENT REGARDING FEDERALLY FUNDED RESEARCH

The U.S. Government has certain rights in this invention based upon partial support by National Institutes of Health Grant Number NS-29632.

### BACKGROUND OF THE INVENTION

Phosphoinositides (“PtdlnsPₙs”) are biosynthesized by the interplay of kinases and phosphatases. These charged lipids are minor components of cellular membranes but are vital as second messengers for diverse cellular functions. PtdlnsPₙs are essential elements in tyrosine kinase, growth factor receptor and G-protein receptor signaling pathways. Furthermore, these lipid signals have important roles in membrane trafficking, including endocytosis, exocytosis, Golgi vesicle movement and protein trafficking, in cell adhesion and migration, in remodeling of the actin cytoskeleton, and in mitogenesis and oncogenesis. Activation of cellular signaling pathways often results from production of one of eight specific PtdlnsPₙs in response to a stimulus, and each PtdlnsPₙ has a specific role for a given signaling pathway in each cell-type.

Phosphoinositide recognition by binding proteins and lipid-metabolizing enzymes involves specific interactions with the phosphoinositide head group and diacylglycerol backbone which vary significantly from protein to protein. When a fluorescent probe is introduced into the inositol head group, binding and metabolism can be attenuated or abrogated entirely. Moreover, many acyl-modified phosphoinositides fail to show adequate Kₘ and Vₘₐₓ values as substrates for lipid kinases and phosphatases. Further, certain phosphoinositide binding proteins demonstrate reduced binding to head group- or acyl-modified phosphoinositides. The simple, robust assays needed for biochemical and cellular studies require a chemically-modified phosphoinositide substrate that can be both acted on by enzymes and recognized by specific binding proteins. A need exists for novel types of phosphoinositides derivatives whose modifications are consistent with the natural binding affinities and sub-cellular localization of the native compounds. Derivatives of this sort would also function as tools useful in drug discovery or drug development assays, and for basic research.

### SUMMARY OF THE INVENTION

The present methods and compositions relate to the fields of pharmacology and drug discovery. More particularly, the methods and compositions concern derivatives of phosphatidylethanolamine-extended phosphoinositides and phosphoinositide polyphosphates (PtdlnsPₙs) and use thereof in drug discovery and development of assays, as well as for basic research purposes. The present invention concerns the design and asymmetric total synthesis of the first examples of a new class of functionalized PtdlnsPₙs, the Pea-PIPₙs.

The synthetic strategy involves homologation of the 1,2-diacylglycerol backbone to a carbon threitol backbone, such as 2,3-diacylthreitol, erythritol or synthetic module. As seen in FIG. 1, such hybrid lipids possess a phosphatidylethanolamine (PE, or Pea) head group at the 1-position and a PtdlnsPₙ head group at the 3,4 and/or 5-position. A reporter group, for example biotin, a fluorophore, or a spin label, may then be covalently attached to the free Pea amino group. FIG. 1 shows an unmodified dipalmitoyl Ptdlns(4,5)P₂ at center, with the acyl-modified NBD (fluorescent N-(7-nitrobenz-2-oxa-1,3-diazol-4-yl)) derivative above and an exemplary Pea-PIPₙ NBD derivative at the bottom. The reporter groups in these synthetic constructs are targeted to the lipid-water interface at a site distant from the key PtdlnsPₙ head group recognition features. The unchanged diacyl moiety permits insertion and retention of Pea-PIPₙs in a lipid bilayer to facilitate recruitment of PtdlnsPₙ-specific binding proteins to a membrane surface environment.

These new hybrid lipids can serve as direct enzymatic substrates that can be delivered into a cell in order to measure direct turnover. This is a great improvement over measuring competitive displacement with a surrogate. Accordingly, Pea-PIPₙs of the present invention have potential for the production of unique, reporter-based high throughput screens or assays for in vitro biochemical activity and for monitoring real time in situ biochemical activity in living cells.

### BRIEF DESCRIPTION OF THE FIGURES AND DRAWINGS

The following figures form part of the present specification and are included to further demonstrate certain aspects of the disclosed embodiments of the invention. The embodiments of the invention may be better understood by reference to one or more of the figures in combination with the detailed description of specific embodiments of the invention presented herein.

FIG. 1. Illustrates (at c) an exemplary hybrid lipid of the present invention that possess a phosphatidylethanolamine (PE, or Pea) head group at the 1-position and a Ptdlns(4,5)P₂ head group at the 4-position in accordance with one embodiment of the present invention. Shown are (a), the unmodified Dipalmitoyl-Ptdlns(4,5)P₂ at the center; (b), the acyl-modified NBD-derivative above (1-C₆-NBD,2-C₆-Ptdlns(4,5)P₂); and (c), the Pea-PIPₙ NBD derivative at the bottom (NBD-Pea-PI(4,5)P₂).

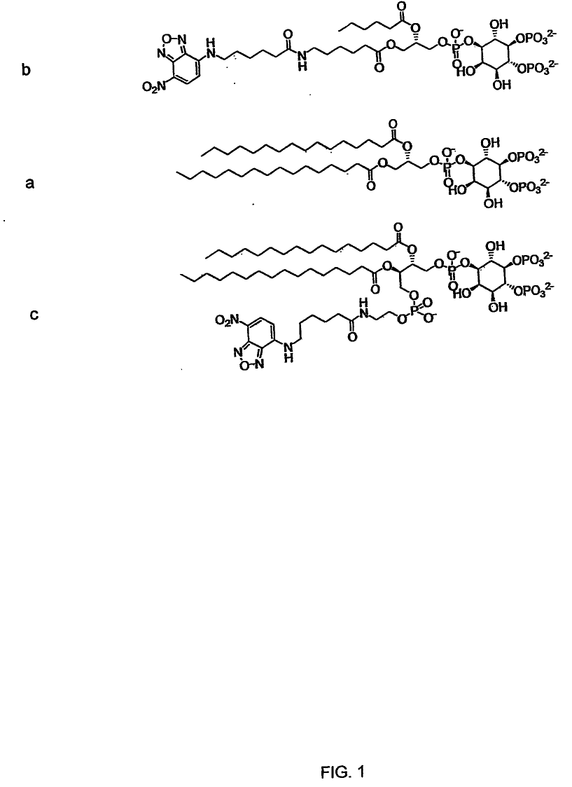

FIG. 2. Illustrates synthesis of exemplary Pea-PIPₙs, including protection debenzylation by hydrogenolysis resulting in each of eight desired Pea-PIPₙs.

FIG. 3. Illustrates exemplary synthesis of differentially functionalized 2,3-diacylthreitol backbones in accordance with an embodiment of the present invention. Reagents and conditions: (a) cyclopentanone, pTSA, toluene, reflux; (b) LiAlH₄, THF; (c) NaH, p-methylbenzyl (PMB)Cl, DMF; (d) 1-H-tetrazole, Cbz-aminoethyl phosphoramidite 4, CH₂Cl₂; (e) 1 M HCl, tetrahydrofuran (THF); (f) C₁₅H₃₁COOH, dicyclohexylcarbodiimide (DCC), DMAP, CH₂Cl₂; (9) dichlorodicyanoquinone (DDQ), CH₂Cl₂/H₂O.

FIG. 4. Illustrates exemplary backbone phosphorylation and synthesis of Pea-PIP₂ derivatives 12. Reagents and conditions: (a) BnOP(NiPr₂)₂, 1-H-tetrazole, CH₂Cl₂; (b) 1-H-tetrazole, 4,5-HG, CH₂Cl₂; (c) H₂ (60 psi), 10% Pd/C, THF/H₂O; (d) probe-NHS ester, 0.5 M TEAB, DMF.

FIG. 5. Illustrates exemplary Pea-PIPₙ reporter groups according to an embodiment of the present invention. Reaction of the free Pea amino group of with four N-hydroxysuccinimidyl (NHS) esters afforded the corresponding biotinylated derivative 4a, the fluorescent N-(7-nitrobenz-2-oxa-1,3-diazol-4-yl) (NBD) and 6-carboxyfluorescein derivatives 4-b and 4-c, and the spin-labeled 3-carboxy-2,2,5,5-tetramethyl-1-pyrrolidinyloxy (PROXYL) derivative 4-d.

FIG. 6. Illustrates an exemplary linker-modified Pea-PIP, analog wherein the linker modification is an amino-PEG-amide.

FIG. 7. Illustrates the synthesis of exemplary short PEG linkers.

FIG. 8. Illustrates the synthesis of an exemplary protected threitol backbone with PEG linkers.

### DETAILED DESCRIPTION

One embodiment of the present invention comprises a strategy for synthesizing a novel class of “two-headed” phospholipid-phosphoinositide hybrids possessing a carbon threitol backbone, such as a 2,3-diacylthreitol, erythritol or synthetic module. These hybrid lipids possess a phosphatidylethanolamine (PE or Pea) head group at the 1-position and a PtdlnsPn head group, such as Ptdlns(4,5)P₂, at the 3,4 and/or 5-position. In particular embodiments, a reporter group, e.g., biotin, a fluorophore, or a chelating agent, may then be covalently attached to the free Pea amino group. The reporter would thus be targeted to the lipid-water interface at a site distant from the key PtdlnsPn head group (for example, Ptdlns(4,5)P₂) recognition features of the binding protein. In various embodiments, the diacyl moiety permits insertion and retention of Pea-PIPₙs in a lipid bilayer to facilitate recruitment of PtdlnsPn head group—(for example, Ptdlns(4,5)P₂—) specific binding proteins to a membrane surface environment.

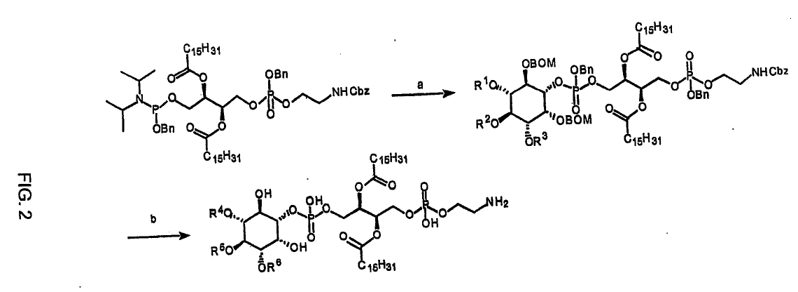

The additional phospholipid head group allows introduction of a biochemical or chemical moiety in a position orthogonal in space to those occupied by the phosphoinositide (“PIPₙ”) head group and the two acyl chains. The method for producing the hybrid phospholipids of the present invention involves diethyl D-tartrate as the chiral precursor for the extended glycerol backbone of the target hybrid lipid. The corresponding acetal is reducted with lithium aluminum hydride and protected with 1 equivalent of PMB-Cl to give the monobenzyl ether. Coupling with a carbonylbenloxy (Cbz)-protected aminoethoxy phosphoramidite yields, after oxidation, the protected phosphatidyl analogue ready for addition of the acyl chains and the selected PIPₙ head group. After acetal hydrolysis, the 2,3-dipalmitoyl derivative is prepared. As shown for exemplary molecules in FIG. 2, the functionalized carbon threitol backbone is then coupled with a phosphatidyl head group. Debenzylation by hydrogenolysis then affords, in particular embodiments, each desired phosphatidylethanolamine-phosphoinositide (“Pea-PIPₙ”) with a free amino groups ready for derivatization.

Any PIPₙ head group may be prepared and used according to the invention. PIPₙ head groups to be utilized in this invention may be commercially obtained or prepared by standard methods known in the art.

In a particular embodiment of the invention, members of this class of molecules have a phosphatidylethanolamine (“Pea”) head group at the 1-position and a phosphoinositide (“PIPₙ”) head group at the 3,4 and/or 5-position.

According to this embodiment, the Pea-diacylthreitol synthetic module is prepared and coupled with any selected phosphoinositide head group. For example, each of eight different naturally occurring phosphoinositide head groups (PI, PI(3)ₚ, PI(4)P, PI(5)P, PI(4,5)P₂, PI(3,4)P₂, PI(3,5)P₂, PI(3,4,5)P₃) has been used to produce Pea-PIPₙs of the present invention (the head groups used for synthesizing Pea-PIPₙs are available commercially, for example from Echelon Biosciences Inc., SLC Utah). Accordingly, certain embodiments of the present invention include Pea-PI, Pea-PI(3)P, Pea-PI(4)P, Pea-PI(5)P, Pea-PI(3,4)P₂, Pea-PI(3,5)P₂, Pea-PI(4,5)P₂ and Pea-PI(3,4,5)P₃.

In another embodiment of the present invention, a linker may be utilized between the PIPₙ headgroup and a reporter. In a particular embodiment, the length of the linker between the PIPₙ headgroup and the reporter moiety at the primary amine is lengthened. In a particular embodiment, the extension for the linker comprises an oligo-polyethylene glycol linker. According to this embodiment, a commercially available di-, tri-, tetra-, and/or penta(ethylene glycol) is used for extending the linker during synthesis of the Pea-PIPₙs.

Other embodiments include additional or alternative linkers. For example, one such alternative linker is a diamino linker that will yield a phosphoramidate final product rather than a phosphate linkage. Another alternative linker uses both phospho- and non-phospho linked spacers. In certain embodiments, this can be accomplished by replacing the Pea group at C-4, such as with a simple ester, amide or other linkage that allows a pendant functionality to be incorporated. Another alternative linker is to use a phosphatidylserine or other carboxylic acid instead of Pea to permit further functionalization at the end distal to the PIPₙ recognition element Various methods for producing alternative linkers are well known in the art.

Pea extension of the invention may also include aminoalcohols as linkers, for example 3-aminopropanol, 4-aminobutanol, and others. In certain embodiments heteroatom-containing derivatives such as 1-amino-11-hydroxy-3,6,9-trixaundecane or similar aminoalcohols with water soluble spacer chains may be used. These can include branched aminoalcohols including, but not limited to, for example, 2-aminomethyl-3-amino-1-propanol which has multiple reactive amino termini for addition of two or more biochemical probes. One of ordinary skill in the art will know methods of Pea extension beyond those listed above.

According to certain embodiments of the present invention, the acyl chains to be attached to any of the above mentioned head groups may include any acyl group from n=2 to n=26 carbons. Methods of modifying the length or degree of double bonds in an acyl chain are well known in the art. In certain embodiments, the carbons will have a number of double bonds, for example from 0 to 6. In alternative embodiments, one or both acyl chains can be replaced with an ether chain of the same length, degree of unsaturation or terminal functionalization. Replacement of one or both acyl chains can be accomplished by any standard method known in the art.

According to other embodiments, the phosphate groups of this invention can be chemically modified to increase stability or resistance to chemical or enzymatic hydrolysis. In certain embodiments, the phosphate groups will be on the inositol, the phosphodiester or the PE phosphodiester. In alternative embodiments, this involves a P═S or P—S bond, replacement of a P—O phosphate linkage with a P—CH₂, P—CHF or P—CF₂ phosphonate linkage or a phosphoramidate linkage. Other methods of chemically modifying the phosphate groups of this invention are known in the art.

In certain embodiments, a Pea-PIPₙ of the present invention has a triester analog at P-1 to allow for an additional site for derivation. Methods of synthesizing a Pea-PIPₙ with a triester analog at P-1 are well known in the art. (See, e.g., Q.-M. Gu and G. D. Prestwich, “Synthesis of Phosphotriester Analogues of the Phosphoinositides Ptdlns(4,5)P2 and Ptdlns(3,4,5)P3,” J. Org. Chem., 61, 8642-8647 (1996)).

In yet another embodiment, the Pea-PIPₙ carbon threitol backbone can include four, five, six or more carbons. In particular embodiments, such carbon backbone may be branched. In a certain embodiment, such carbon threitol backbone is 2,3-diacylthreitol or erythritol.

In yet another embodiment, Pea-PIPₙs include a polymerizable group that allows for the construction of Pea-PIPₙs polymers. Accordingly, particular embodiments of the invention comprise oligomeric Pea-PIPₙs formed by linking two or more Pea-PIPₙ molecules together.

In particular embodiments, a reporter group (or “label” or “tag”) is covalently attached to the free Pea amino group. Such a reporter may include, for example, a fluorescent label, a radiolabel, a chemiluminescent label, a spin label, a photophore, a chromophore, biotin, a nanogold particle, and/or any suitable reporter, and mixtures thereof.

As used herein, suitable fluorescent compounds that can be used according to the present invention include chemically activated, thetherable analogs of acrylodan, AMCA, BODIPY, Cascade-Blue, CINERF, dansyl, dialkylaminocoumarin, eosin, erythrosine, fluorescein (FITC), hydroxycoumarin, NBD, Oregon green, PyMPO, pyrene, rhodamine, Rhodol Green, TMR, Texas Red, X-Rhodamine, and the like.

Attached to the Pea amino group, the reporter would thus be targeted to the lipid-water interface at a site distant from the specific features of the PtdlnsPₙ head group necessary for interaction with lipid recognition proteins or other chemicals or compounds that specifically interact with the PtdlnsPₙ at a membrane surface. In various embodiments, the diacyl moiety permits insertion and retention of Pea-PIPₙs in a lipid bilayer to facilitate recruitment of PtdlnsPₙ-specific binding proteins to a membrane surface environment. An individual Pea-PIPn may have one or more reporters which may be of the same or different types. In various embodiments, the Pea-PIPns will be transported into cells, with or without a reporter.

All stereoisomers, which include enantiomers or diasteromers, for any component of the Pea-PIPₙ molecules can be employed in any embodiment of this invention. Such modifications are well known in the art.

Certain embodiments will include Pea-PIPₙs bound to a surface, for example for use in a biochemical assay. In particular embodiments, such surface will include a plate, a bead or nitrocellulose. In a particular embodiment, such surfaces are selected from the group consisting of, but not limited to, a chemically activated glass, plastic or other surface; activated agarose, polystyrene or any other type of bead and nitrocellulose. In other embodiments, the Pea-PIPₙs are attached to a metal surface, such as gold. In particular embodiments, attachment to gold is accomplished by introducing to the Pea-PIPₙ a pendant alkyl thiol moiety that is capable of attaching to a gold surface. Methods for making such pendant alkyl thiol derivatives are well known in the art.

In alternative embodiments, the Pea-PIPₙs will be incorporated in a liposome. Such liposome incorporated Pea-PIPₙs may include a reporter.

Certain embodiments allow for the use of Pea-PIPₙs for assays. The compositions and methods of their use can be used in any assay that currently uses a modified Pea-PIP. Particular embodiments include in vitro fluorogenic, FRET, ELISA and chemiluminsence assays. These may be in a high-throughput format. Alternative embodiments include in vitro enzyme assays, lipid kinase or phosphatase activity, cell-based assays and agonist or antagonist assays.

Such assays include, but are not limited to, in vitro enzyme assays, in vitro agonist or antagonist assays or cell-based assays. In some embodiments of the invention, labeled Pea-PIPₙs can also be linked or bound to plates, beads or other surfaces which may, in particular embodiments, be coated with a means for binding Pea-PIPₙ thereto. Labels of use in the present invention may be activated by any method known in the art in order to effect attachment to a Pea-PIPₙ of the present invention. Methods of use in the attachment of a label include, but are not limited to, the activation of an ester, carbonyldiimidazole activation and use of any Michael acceptor, such as acrylates, acrylamide, maleimides, vinylsulfone, a,b-unsaturated ketones, esters, aldhydes, amides, and the like.

An example of such a means of linking or binding a Pea-PIPₙs is streptavidin. Another example is NHS activation. In other embodiments, the Pea-PIPn is bound to nitrocellulose. In particular embodiments, the reporter is biotin or a fluorescent label. Fluorescent compounds suitable for use as a label or reporter according to the present invention include, but are not limited to, chemically activated tetherable analogs of acrylodan, AMCA, BODIPY, Cascade-Blue, CINERF, dansyl, dialkylaminocoumarin, eosin, erythosine, fluorescein (FITC), hydroxycoumarin, NBD, Oregon green, PyMPO, pyrene, rhodamine, Rhodol Green, TMR, Texas Red, X-Rhodamine, and the like.

One embodiment of the present invention provides a method of screening for phosphoinositide-specific binding proteins in a membrane surface environment.

In particular embodiments a high throughput screen (HTS) comprising compositions of the invention can be used for identifying, for example, chemicals, natural products and/or or synthetic compounds that affect phosphoinositide recognition and/or signaling at a cell membrane. Such compounds include, but are not limited to, for example, agonists and antagonists for protein kinases and phosphoinositide kinases and for phosphoinositide and inositol phosphate binding proteins that are regulated by PIPₙs or IPₙs and may serve as downstream effectors in signaling pathways important for therapeutic interventions. In particular embodiments, lipid phosphatases or phospholipases are identified. In alternative embodiments, HTS assays include cell-based assays using intracellular PIPₙs introduced by the shuffling system and can use primary cells, immortalized cells, cancer cells, cells transformed with plasmids encoding key enzymes or other proteins, and the like. The assays could also use in vitro cell extracts or partially purified or homogeneous proteins.

In other embodiments the Pea-PIPₙs are introduced into cells. Such introduced Pea-PIPₙs may be labeled, or tagged, with one or more reporters. In a particular embodiment, fluorescent acyl-modified Pea-PIPₙs are shuttled into cells where they exhibit subsequent appropriate membrane localization.

In certain embodiments, assays according to the present invention are performed in living cells. Particular embodiments of the invention provide compositions and methods for visualizing the location of labeled phosphoinositides within a cell. A method for facilitating uptake of a Pea-PIPₙs into a cell comprises contacting the cell with a composition of matter comprising a Pea-PIPₙ and a shuttle, or other method of introducing the Pea-PIPₙ into the cell. Compounds suitable for shuttling Pea-PIPₙs into a cell can include, but are not limited to, polyamines. Such polyamines can include, for example, aminoglycosidic aminocyclitols (e.g., aminoglycoside antibiotics), synthetic “spherical” dendrimeric polyamines, polybasic nuclear proteins (histones), polybasic polypeptides, lipidic polyamines, polyethyleneimine, steroidal polyamines, and the like, and mixtures thereof. Other polybasic proteins (or polybasic polypeptides) useful for introducing a Pea-PIPₙ into a cell include proteins or polypeptide that contains sufficient lysine, arginine, and/or histidine residues to complex an anionic ligand, such as an Pea-PIPₙ. The polybasic polypeptide may also contain unnatural or non-protein amino acids, N-acylglycine groups, and any of a known group of amide group replacements known as peptide bond isosteres. In particular embodiments, assays performed in living cells are monitored by high-content screening methods or confocal microscopy. Such in vitro assays allow for minimal disruption of the normal cellular environment

The disclosed compositions and methods of their use can be used for the discovery of new pharmaceutical agents and targets related to PtdlnsPₙs compounds.

The skilled artisan will realize that the chemical modifications listed above are exemplary only and that many variations may be used, depending on the particular type of Pea-PIP, to be synthesized.

Definitions

For the purposes of the present invention, the following terms shall have the following meanings:

“Tag” or “labels” are used interchangeably to refer to any atom, molecule, compound or composition that can be used to identify a Pea-PIP, to which the label is attached. In various embodiments of the invention, such attachment may be either covalent or non-covalent In certain embodiments of the invention, the labels have physical characteristics that facilitate the identification of the label. In non-limiting examples, labels may be fluorescent, phosphorescent, luminescent, electroluminescent, chemiluminescent or any bulky group or may exhibit Raman or other spectroscopic characteristics. It is anticipated that virtually any technique capable of detecting and identifying a labeled nucleotide may be used, including visible light, ultraviolet and infrared spectroscopy, Raman spectroscopy, nuclear magnetic resonance, electron paramagnetic resonance, positron emission tomography, scanning probe microscopy and other methods known in the art.

Moreover, for the purposes of the present invention, “a” or “an” entity refers to one or more of that entity; for example, “a Pea-PIP” or “a Pea-PIPₙ” refers to one or more of the compound or at least one compound. As such, the terms “a” or “an”, “one or more” and “at least one” can be used interchangeably herein. It is also noted that the terms “comprising,” “including,” and “having” can be used interchangeably.

Furthermore, a compound “selected from the group consisting of” refers to one or more of the compounds in the list that follows, including mixtures (i.e. combinations) of two or more of the compounds.

According to the present invention, an isolated or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

As used herein, “shuttle” means a compound, polymer, complex, or mixture thereof that facilitates transport of phosphoinositides, inositol polyphosphates, and mixtures thereof into cells. Preferred shuttles comprise polyamines.

### EXAMPLES

It should be appreciated by those skilled in the art that the techniques disclosed in the examples which follow represent techniques discovered by the inventors to function well in the practice of the invention, and thus can be considered to constitute the preferred modes for its practice. However, those of skill in the art should appreciate, in light of the present disclosure, that many changes can be made in the specific embodiments disclosed herein which will still obtain a like or similar result without departing from the spirit and scope of the invention.

### Example 1

### Synthesis of Functionalized Phosphoinositide Polyphosphates, the Pea-PIPₙs, and Reporter Analogs

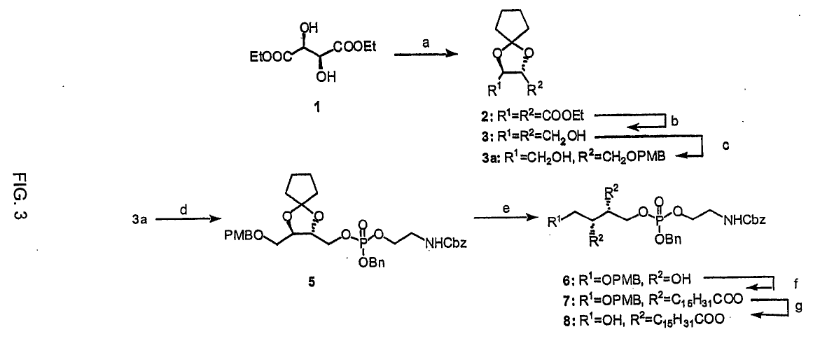

The general method for synthesis of Pea-PIPₙs of the present invention is described in Rzepecki, P. W. and Prestwich, G. D.: J. Org. Chem. 2002, 67(16): 5454-60, which is hereby incorporated by reference in its entirety. In the steps disclosed below, the numbers in bold refer- to compounds and synthetic intermediates shown in FIGS. 3, 4 and 5.

Diethyl D-tartrate was chosen as the chiral precursor for the extended glycerol backbone of the target hybrid lipid. The absolute configuration of both stereogenic centers at C-2 and C-3 is identical to the configuration of glycerol sn-2 position in naturally-occurring PtdlnsPₙs and in natural Pea. Moreover, the C₂ axis allowed the use of a monoprotection step in the early stages of the synthesis. Synthesis of an exemplary embodiment, Pea-PI(4,5)P₂ and reporter analogs is shown in FIGS. 3, 4 and 5. Diethyl D-tartrate 1 was protected as a cyclopentylidene acetal, which was found to be most readily removed after backbone functionalization. Initially, an isopropylidene acetal was used, but scale-up of the deprotection led to unsatisfactory yields of the desired intermediate. Reduction of acetal 2 with lithium aluminum hydride afforded (2R,3R)-O-cyclopentylidene threitol 3a, which was protected with 1 equiv. of PMB-Cl to give the monobenzyl ether 3b. The primary alcohol of 3b was converted to a Cbz-protected Pea head group by coupling to the phosphoramidite 4, which after oxidation afforded the protected PE analogue 5. Acidic hydrolysis yielded diol 6 in 65% yield after silica chromatography. Acylation with palmitic acid provided diester 7, and oxidative cleavage of the p-methoxybenzyl (PMB) with dichlorodicyanoquinone (DDQ) gave primary alcohol 8.

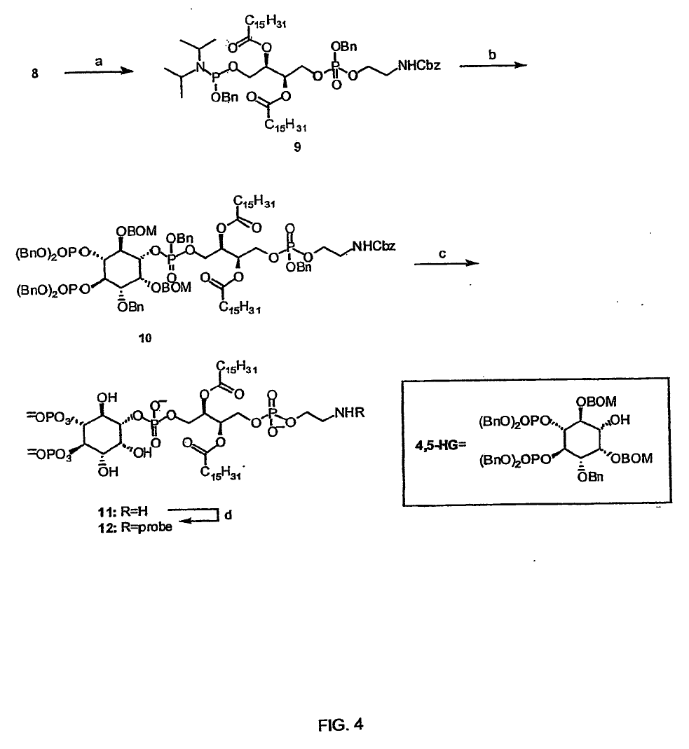

FIG. 4 illustrates the installation of two different phosphorylated head groups on the 2,3-diacylthreitol backbone. Thus, reaction of alcohol 8 with benzyltetraisopropylphosphordiamidite yielded a homologated Pea-like phosphoramidite reagent 9, which was coupled with the protected myoinositol 4,5-bisphosphate head group obtained as previously described (Prestwich, G. D.; Chaudhary, A; Chen, J.; Feng, L; B. Mehrotra; Peng, J. In Phosphoinositides: Chemistry, Biochemistry and Biomedical Applications; Bruzik, K. S., Ed.; American Chemical Society: Washington, D.C., 1999; Vol. 818, p 24-37), to give the fully protected Pea-PIP₂ precursor 10. Global debenzylation of 10 was accomplished by hydrogenolysis to give the free phosphate monoesters and phosphodiesters in the hybrid lipid Pea-PIP₂ (11). Reaction of the free amino group of 11 with four succinimidyl esters afforded the corresponding biotinylated derivative 12a, the fluorescent NBD and 6-carboxyfluorescein derivatives 12b and 12c, and the spin-labeled PROXYL (tetramethyl-1-pyrrolidinyloxy) derivative 12d. Biological results are described below for biotinylated derivative 12a and fluorescent analogue 12c. The spin-labeled derivative 12d may be used to probe interfacial protein-lipid interactions in liposomes, in analogy to the use of acyl spin-labeled probes to characterize the MARCKS peptide-Ptdlns(4,5)P₂ interaction in liposomes.

The steps comprising an exemplary synthetic process resulting in the novel hybrid lipid, Pea-PI(4,5)P₂, are described in detail as follows:

(2R,3R)-1,4-Dioxa-spiro[4.4]nonane-2,3-dicarboxylic acid diethyl ester 2. Diethyl D-tartrate 1 (1.004 g, 4.87 mmol), cyclopentanone (2.2 mL, 24.35 mmol, 5 equiv.) and p-toluenesulfonic acid (93 mg, 0.49 mmol, 0.1 equiv.) were dissolved in toluene (75 mL) and stirred under reflux for 36 h with azeotropic removal of water using a Dean-Stark trap. Upon completion, the reaction mixture was cooled to room temperature (rt) and neutralized with solid NaHCO₃. Solid salts were filtered off, the filtrate was concentrated in vacuo, and the crude product was purified on SiO₂ (hexane:acetone 4:1 containing 10% v/v Et₃N) to give 1.166 g (4.28 mmol, 88%) of acetal 2 as a colorless oil. ¹H NMR (400 MHz, CDCl₃) □ 4.73 (s, 2H), 4.28 (q, 4H, J=5.4), 1.94-2.04 (m, 2H), 1.80-1.90 (m, 2H), 1.65-1.76 (m, 4H), 1.32 (t, 6H, J=5.4); ¹³C NMR (100 MHz, CDCl₃) δ 169.67, 123.34, 77.08, 61.85, 36.66, 23.50, 14.15; IR: 2980, 1756, 1337, 1119, 1115, 1023, 460, 453. Anal. Calcd for C₁₃H₂₀O₆: C, 57.34; H, 7.40. Found: C, 57.34; H, 7.21.

(2R,3R)-(3-Hydroxymethyl-1,4-dioxa-spiro[4.4]non-2-yl)-methanol 3a. A solution of diethyl ester 2 (1165 mg, 4.28 mmol, 1 equiv.) in dry tetrahydrofuran (THF) was transferred via canula to a suspension of LiAlH₄ (244 mg, 6.42 mmol, 1.5 equiv.) in dry THF, pre-cooled in brine/ice bath. The reaction mixture was stirred at rt for 24 h and then saturated aq. potassium sodium tartrate was added dropwise to decompose excess hydride reagent The mixture was stirred for additional 24 h, and then extracted with three portions of CH₂Cl₂. The combined organic phases were dried over MgSO₄, concentrated in vacuo, and the crude product was purified on SiO₂ (hexane:acetone 3:2 containing 10% v/v Et₃N) to give 805 mg (4.27 mmol, 99%) of the diol 3a as a colorless oil. ¹H NMR (400 MHz, CDCl₃) δ 3.85-3.95 (m, 2H), 3.60-3.80 (m, 4H), 1.73-1.85 (m, 4H), 1.60-1.73 (m, 4H); ¹³C NMR (100 MHz, CDCl₃) δ 119.36, 78.40, 62.46, 37.32, 23.44; IR: 3390, 2956, 2875, 1434, 1335, 1204, 1112, 1041, 973. Anal. Calcd for C₉H₁₆O₄: C, 57.43; H, 8.57. Found: C, 57.48; H, 8.52.

(2R,3R)-[344-Methoxy-benzyloxymethyl)-1,4-dioxa-spiro[4.4]non-2-yl-methanol 3b. To a suspension of NaH (175 mg, 4.38 mmol, 1 equiv.) in dry DMF was added alcohol 3a (824 mg, 4.38 mmol, 1 equiv.) in dry DMF via canula. The mixture was cooled to 0° C. and then 4-methoxybenzyl chloride (0.65 ml, 4.82 mmol, 1.1 equiv.) was added dropwise over 20 min. The ice bath was removed and the mixture was stirred at rt for 18 h. Traces of NaH were decomposed by slow addition of water. The mixture was extracted 3 times with CH₂Cl₂, dried (MgSO₄), and concentrated in vacuo. The crude product was purified on SiO₂ (hexane:acetone 4:1 containing 10% v/v Et₃N) to give 879 mg (2.85 mmol, 65%) of compound 3a as a colorless oil. ¹H NMR (400 MHz, CDCl₃) δ 7.20-7.30 (m, 2H), 6.80-6.95 (m, 2H), 4.45-4.55 (m, 2H), 3.92-4.00 (m, 1H), 3.82-3.89 (m, 1H), 3.79 (s, 3H), 3.60-3.76 (m, 3H), 3.49 (dd, 1H. J₁=4.7, J₂=7.4), 2.45 (bs, 1H), 1.73-1.89 (m, 4H), 1.58-1.73 (m, 4H); ¹³C NMR (100 MHz, CDCl₃) δ 159.29, 129.70, 129.40, 128.52, 119.31, 113.82, 79.56, 76.58, 73.21, 70.14, 62.63, 55.19, 37.26, 37.20, 23.52, 23.40. IR: 3466, 2955, 2872, 1612, 1514, 1248, 1101, 1035. Anal. Calcd for C₁₇H₂₄O₅: C, 66.21; H, 7.84; Found: C, 66.27, H, 7.76.

[2-(Benzyloxy-diisopropylamino-phosphanyloxy)-ethyl]-carbamic acid benzyl ester 4. To a solution of benzyloxybis(N,N-diisopropylamino)phosphine (1.538 g, 4.54 mmol, 1.5 equiv.) and 1-H-tetrazole (106 mg, 1.51 mmol, 0.5 equiv.) in dry CH₂Cl₂, was added a solution of (2-hydroxyethyl)carbamic acid benzyl ester (591 mg, 3.03 mmol) in CH₂Cl₂. The mixture was stirred at rt for 3 h, concentrated in vacuo, and the crude product was purified on SiO₂ (hexane:acetone:Et₃N 6:4:1) to give 886 mg (2.05 mmol, 68%) of compound 4 as an air- and moisture-sensitive colorless oil. ¹H NMR: (400 MHz, CDCl₃) δ 7.20-7.40 (m, 10H), 5.21 (bs, 1H), 5.08 (s, 2H), 4.69 (m, 2H), 3.64-3.79 (m, 2H), 3.56-3.70 (m, 2H), 3.39 (m, 2H), 1.18 (d, 12H, j=5.4). ³¹P NMR (162 MHz, CDCl₃): δ 149.1.

(2-Benzyloxy-[(2R,3R)-3-(4-methoxy-benzyloxymethyl)-1,4-dioxa-spiro[4.4]non-2-ylmethoxy]-phosphoryloxy)ethyl)-carbamic acid benzyl ester 5. A solution of the monoprotected alcohol 3b (486 mg, 1.58 mmol, 1 equiv.) and tetrazole (331 mg, 473 mmol, 3 equiv.) in dry CH₂Cl₂ (5 mL) was stirred under N₂ for 5 min at rt. Phosphoramidite 4 (886 mg, 2.05 mmol, 1.3 equiv.) in dry CH₂Cl₂ (5 mL) was added via canula, and the mixture was stirred for 2 h (until all starting material was consumed). After cooling to 40° C., mCPBA (1.360 g, 6 mmol, 3 equiv., 60%) in CH₂Cl₂ (5 mL) was added and the reaction mixture was stirred for 5 min. The cold bath was then removed and stirring was continued for an additional 1 h. The reaction was diluted with CH₂Cl₂ and poured into satd. NaHCO₃, and after 20 min of vigorous stirring extracted 3 times with CH₂Cl₂. Combined organic phases were dried (MgSO₄), concentrated in vacuo, and the crude product was purified on SiO₂ (hexane:acetone 3:2) to give 1.033 g (1.57 mmol, 99%) of compound 5 as a colorless oil. ¹H NMR (400 MHz, CDCl₃) δ 7.28-7.38 (m, 10H), 7-18-7-24 (m, 2H), 6.82-6.88 (m, 2H), 5.44-5.54 (m, 1H), 5.00-5.12 (m, 4H), 4.42-4.51 (m, 2H), 3.99-4.17 (m, 4H), 3.89-3.99 (m, 2H), 3.76 (s, 3H), 3.56 (dd, 1H, J₁=3.6, J₂=7.5), 3.43-3.50 (m, 1H), 3.33-3.41 (m, 2H), 1.70-1.84 (m, 4H), 1.54-1.70 (m, 4H); ¹³C NMR (100 MHz, CDCl₃) □ 159.30, 156.34, 136.47, 135.65, 135.58, 129.92, 129.79, 129.35, 128.68, 128.63, 128.48, 128.35, 128.07, 128.03, 119.95, 113.80, 77.24, 76.00, 73.18, 69.95, 69.53, 67.46, 66.94, 66.71, 55.21, 53.47, 41.30, 37.26, 37.20, 23.49, 23.42; ³¹P NMR (162 MHz, CDCl₃): δ 2.23; IR: 3316, 2956, 1722, 1514, 1250, 1023. Anal. Calcd for C₃₄H₄₂NO₁₀P: C, 62.28; H, 6.46; N, 2.14. Found: C, 62.38; H, 6.29; N, 2.18.

(2-{Benzyloxy-[(2R,3R)-2,3-dihydroxy-4-(4-methoxy-benzyloxy)-butoxy]-phosphoryloxy}-ethyl)-carbamic acid benzyl ester 6. To a solution of 5 (2.161 g, 3.3 mmol) in dry THF (100 mL) was added 100 mL of 1 M HCl. The mixture was stirred at rt for 3 h and then satd. aq. NaHCO₃ was added. The mixture was transferred to a separatory funnel and extracted with CH₂Cl₂. The crude product was purified on SiO₂ (toluene:acetone 1:4) to give 1.267 g (2.15 mmol, 65%) of the diol 6 as a colorless oil. ¹H NMR (400 MHz, CDCl₃) δ 7.25-7-35 (m, 10H), 7.17-7.22 (m, 2H), 6.81-6.86 (m, 2H), 5.64-5.75 (m, 1H), 4.95-5.10 (m, 4H), 4.35-4.46 (m, 2H), 3.95-4.12 (m, 4H), 3.78-3.86 (m, 1H), 3.65-3.78 (m⁻¹H), 3.75(s, 3H), 3.45-3.55 (m, 2H), 3.30-3.40 (m, 2H); ¹³C NMR (100 MHz, CDCl₃) □ 159.31, 156.51, 136.45, 135.50, 129.77, 129.42, 128.73, 128.64, 128.48, 128.06, 113.83, 73.14, 71.24, 70.50, 69.65, 69.25, 68.90, 66.95, 66.73, 55.22, 41.24; ³¹P NMR (162 MHz, CDCl₃) δ 0.52; IR: 3349, 2954, 1719, 1612, 1513, 1456, 1248, 1023, 821, 739, 698. Anal. Calcd for C₂₉H₃₆NO₁₀P: C, 59.08; H, 6.15; N, 2.38; 0, 27.14; P, 5.25. Found: C, 58.96; H, 6.27; N, 2.44, P, 5.44.

Hexadecanoic acid {(2R,3R)-3-[benzyloxy-(2-benzyloxycarbonylamino-ethoxy)-phosphoryloxy]-2-hexadecanoyloxy-1-(4-methoxy-benzyloxymethyl)}-propyl ester 7. DCC (1.377 g, 6.67 mmol, 3 equiv.), and dimethylaminopyridine (DMAP) (299 mg, 2.45 mmol, 1.1 equiv.) were added in one portion to a solution of 6 (1.311 g, 2.22 mmol, 1 equiv.) and hexadecanoic acid (1.901 g, 6.67 mmol, 3 equiv.) in dry CH₂Cl₂. After stirring for 18 h, the reaction mixture was concentrated in vacuo and purified on SiO₂ (hexane:acetone 4:1) to give 1.68 g (1.58 mmol, 71%) of product 7 as a waxy solid, mp 60° C. ¹H NMR (400 MHz, CDCl₃) □ 7.26-7.40 (m, 10H), 7.18-7.24 (m, 2H), 6.82-6.88 (m, 2H), 5.08 (s, 2H), 4.95-5.06 (m, 2H), 4.32-4.46 (m, 2H), 3.88-4.24 (m, 4H), 3.76 (s, 3H), 3.43-3.60 (m, 2H), 3.34-3.43 (m, 2H), 2.21-2.32 (m, 4H), 1.50-1.64 (m, 4H), 1.25 (bs, 48H), 0.88 (t, 6H, J=5.3); ¹³C NMR (100 MHz, CDCl₃) δ 172.90, 172.77, 159.35, 129.41, 128.73, 128.65, 128.47, 128.08, 128.01, 113.80, 72.97, 70.04, 69.97, 69.74, 69.66, 69.62, 67.45, 67.02, 66.71, 65.65, 55.18, 41.30, 34.15, 31.94, 29.72, 29.67, 29.51, 29.38, 29.31, 29.15, 24.92, 24.86, 22.70, 14.12; ³¹P NMR (162 MHz, CDCl₃) δ 0.26, 0.14. IR: 2924, 2853, 1741, 1612, 1514, 1456, 1249, 1154, 1112, 1035, 736, 697. Anal. Calcd for C₆₁H₉₆NO₂P: C, 68.70; H, 9.07; N, 1.31. Found: C, 68.87; H, 8.81; N, 1.32.

Hexadecanoic acid {(2R,3R)-3-benzyloxy-(2-benzyloxycarbonylamino-ethoxy)-phosphoryloxy]-2-hexadecanoyloxy-1-hydroxymethyl}-propyl ester 8. To a solution of 7 (234 mg, 0.23 mmol, 1 equiv.) in CH₂Cl₂ (23 mL) was added water (0.23 mL) followed by DDQ (103 mg, 0.46 mmol, 2 equiv.). When TLC indicated that the reaction was complete, the mixture was transferred to separatory funnel and washed with 5% Na₂SO₃ and satd. NaHCO₃ (2×). The aqueous phases were back-extracted once with CH₂Cl₂, and the combined organic phases were dried (MgSO₄), concentrated in vacuo, and product was purified on SiO₂ (hexane:acetone 4:1) to give 125 mg (0.13 mmol, 60%) of product 8 as a waxy solid, mp ˜60° C. ¹H NMR (400 MHz, CDCl₃) δ 7.20-7.40 (m, 10H), 5.0-5.16 (m, 6H), 3.92-4.23 (m, 4H), 3.53-3.72 (m, 2H), 3.30-3.45 (m, 2H), 2.80-2.94 (m, 1H), 2.27-2.33 (m, 4H), 1.54-1.64 (m, 4H), 1.25 (bs, 48H), 0.88 (t, 6H, J=5.3); ¹³C NMR (100 MHz, CDCl₃) δ 173.44, 173.16, 156.46, 136.45, 135.46, 128.81, 128.70, 128.51, 128.13, 128.06, 71.64, 70.05, 69.81, 67.08, 66.80, 65.79, 60.70, 41.30, 34.15, 31.94, 29.73, 29.68, 29.52, 29.38, 29.31, 29.16, 24.90, 22.70, 14.13; ³¹P NMR (162 MHz, CDCl₃) □ 1.68, 1.61; IR: 2917, 2850, 1739, 1467, 1263, 1017. Anal. Calcd for C₅₃H₈₈NO₁₁P: C, 67.27; H, 9.37; N, 1.48. Found: C, 67.32; H, 9.49; N, 1.50.

Hexadecanoic acid {(2R,3R)-1-[benzyloxy-(2-benzyloxycarbonylamino-ethoxy)-phosphoryloxymethyl]-3-(benzyloxy-diisopropylamino-phosphanyloxy)-2-hexadecanoyloxy}-propyl ester 9. To a solution of benzyltetraisopropylphosphordiamidite (1.164 g, 3.44 mmol, 1.5 equiv.) and tetrazole (80 mg, 1.15 mmol, 0.5 equiv.) in dry CH₂Cl₂ a solution of ester 8 (2.169 g, 2.30 mmol, 1 equiv.) in CH₂Cl₂ was added via canula. After 2 h, the reaction was concentrated in vacuo and the residue was purified on SiO₂ (ethyl acetate:toluene 4:1 containing 5% EtN) to give 2.190 g (1.85 mmol, 81%) of phosphoramidite 9. ¹H NMR (400 MHz, CDCl₃) δ 7.20-7.40 (m, 10H), 5.0-5.16 (m, 6H), 3.92-4.23 (m, 4H), 3.53-3.72 (m, 2H), 3.30-3.45 (m, 2H), 2.80-2.94 (m, 1H), 2.27-2.33 (m, 4H), 1.54-1.64 (m, 4H), 1.25 (bs, 48H), 0.88 (t, 6H, V-5.3). ¹³C NMR (100 MHz, CDCl₃) δ 172.91, 172.75, 139.18, 135.58, 135.52, 128.74, 128.67, 128.49, 128.31, 128.26, 128.09, 128.02, 127.36, 127.29, 126.96, 126.93, 70.59, 69.72, 67.01, 66.72, 65.70, 65.37, 65.20, 61.69, 43.10, 34.18, 31.95, 29.73, 29.68, 29.52, 29.38, 29.33, 29.17, 24.88, 24.58, 22.70, 14.13; ³¹P NMR (162 MHz, CDCl₃) δ 150.18, 149.79, 0.32, 0.22.

Hexadecanoic acid 1-[benzyloxy-2-benzyloxycarbonylamino-ethoxy)-phosphoryloxymethyl]-2-hexadecanoyloxy-3[-benzyloxy-phosphoryloxy-3-benzyloxy-2,6-bis(benzyloxymethoxy)-4,5-bis-(bis-benzyloxy-phosphoryloxy)-D-myo-inositol]-propyl ester 10. To a solution of 4,5-head group (4,5-HG, 250 mg, 0.24 mmol, 1 equiv.) and tetrazole (51 mg, 0.73 mmol, 3 equiv.) in dry CH₂Cl₂ was added a solution of phosphoramidite 9 (345 mg, 0.29 mmol, 1.2 equiv.) in CH₂Cl₂. The mixture was stirred at rt for about 3 h, cooled to −40° C. and mCPBA (210 mg, 0.73 mmol, 3 equiv., 60%) was added. After 15 min, the cold bath was removed and stirring was continued for additional 1 h. The reaction mixture was diluted with CH₂Cl₂ and poured into a solution of 5% Na₂SO₃ and satd. NaHCO₃. The mixture was extracted 3 times with CH₂Cl₂ and combined organic fractions were dried over MgSO₄, concentrated in vacuo, and purified on SiO₂ (hexane:acetone 3:2) to give 306 mg (0.14 mmol, 59%) of product 10 as a colorless oil. ¹H NMR (400 MHz, CDCl₃) □ 7.00-7.35 (m, 50H), 5.55-5.95 (m, 1H), 5.15-5.35 (m, 2H), 4.40-5.15 (m, 27H), 4.20-4.40 (m, 2H), 3.90-4.20 (m, 6H), 3.45-3.60 (m, 1H), 3.25-3.45 (m, 2H), 2.10-2.30 (m, 4H), 1.40-1.60 (m, 4H), 1.05-1.35 (m, 48H), 0.88 (t, 6H, J=5.3); ¹³C NMR (100 MHz, CDCl₃) δ 172.59, 138.09, 137.71, 137.25, 136.87, 136.56, 136.23, 136.17, 136.10, 136.06, 135.99, 135.90, 135.82, 135.51, 135.54, 128.65, 128.44, 128.34, 128.26, 128.02, 127.84, 127.75, 127.42, 96.64, 95.46, 78.99, 77.61, 77.34, 76.93, 76.60, 74.89, 72.79, 72.02, 70.45, 69.90, 69.70, 69.44, 69.12, 66.97, 66.62, 65.20, 41.29, 33.97, 31.92, 29.70, 29.67, 29.66, 29.51, 29.35, 29.29, 29.13, 24.78, 22.68, 14.12; ³¹P NMR (162 MHz, CDCl₃) δ 0.35, −0.15, −0.56 (ratio 1:2:1). IR: 2924, 2853, 1743, 1455, 1273, 1022. Anal. Calcd for Cl₁₇H₁₅₃NO₂₇P₄: C, 65.99; H, 7.24; N, 0.66; P, 5.82. Found: C, 65.75; H, 7.24; N, 0.73; P, 6.12.

1-[(2R,3R)-4-(2-Aminoethoxyphosphoryloxy)-2,3-di-O-palmitoylbutoxyphosphoryloxy]-4,5-myo-bisphosphate 11. To a solution of compound 10 (193.4 mg, 0.091 mmol) in a mixture of THF/water (4:1, v/v, 50 mL) was added 10% palladium on charcoal (387 mg). The mixture was shaken for 18 h at rt under 60 psi of H₂. The catalyst was removed by filtration and solvent was removed in vacuo. The crude product was redissolved in water and stirred for 3 h with Dowex 50×-100 resin (Na⁺ form). The resin was removed by filtration and the filtrate was lyophilized to give 77.3 mg (0.062 mmol, 62%) as the sodium salt. The dried crude product was used for coupling with activated (N-hydroxysuccinimidyl, or “NHS”) esters as described below. ¹H NMR (400 MHz, D₂O): □5.15-5.30 (m, 2H), 4.10-4.30 (m, 2H), 3.85-4.10 (m, 7H), 3.75-3.85 (m, 1H), 3.6-3.7 (m, 1H), 3.4-3.5 (m, 1H), 3.18 (bs, 2H), 2.1-2.5 (m, 4H), 1.4-1.6 (bs, 4H), 1.18 (bs, 48H), 0.76 (bs, 6H). ³¹P NMR (162 MHz, D₂O): δ 2.36, 1.80, 1.09, 0.56 (ratio 1:1:1:1), MS MALDI (free acid): 146 (M+Na), 950 (M+3Na—C₁₅H₃₁CO), 928 (M+2Na—C₁₅H₃₁CO), 906 (M+Na—C₁₅H₃₁CO), 884 (M—C₁₅H₃₁CO).

General procedure for coupling with NHS esters. To a solution of compound 11 (˜10 μmol, 1 equiv.) in 0.5 M TEAB (0.5 mL, pH 7.5) was added a solution of appropriate NHS ester (˜12 □mol, 1.2 equiv.) (three of which were obtained from Molecular Probes, Inc.) in 0.5 mL of dimethylformamide (DMF(was added. PROXYL-SE was prepared as described in Rauch, M.; Ferguson, C.; Prestwich, G. D.; Cafiso, D. J. Biol. Chem. 2002. The mixture was stirred at rt for 18 h, and solvents were then removed in vacuo. The residue was washed 4 times with acetone and then purified on DEAE-cellulose column with a step gradient of TEAB (0 to 2 M). The desired fractions were pooled, lyophilized, converted by ion exchange into a sodium salt, and lyophilized again.

Biotin derivative 12a. Reaction of 11 (9.4 mg, 7.5 mmol) with Biotin-X, SE (4.4 mg, 9.7 mmol) yielded 6.4 mg (4 mmol, 53%) of 12a. ¹H NMR (400 MHz, D₂O) δ 5.20-5.40 (m, 4H), 4.50-4.60 (m, 1H), 4.30-4.45 (m, 1H), 3.70-4.45 (m, 9H), 3.60-3.70 (m, 1H), 3.30-3.45 (m, 1H), 3.05-3.20 (m, 4H), 2.65-2.95 (m, 2H), 2.00-2.50 (m, 8H), 1.40-1.80 (m, 12H), 0.90-1.40 (m, 52H), 0.70-0.90 (m, 6H); ³¹P NMR (162 MHz, D₂O δ 3.22, 2.32, 1.34, 0.41 (ratio 1:1:1:1). MS MALDI (free acid): 1528 (M+3Na), 1506 (M+2Na), 1484 (M+Na), 1462 (M−H), 1223 (M−H—C₁₅H₃₁CO), 1122 (M−H-biotin). HR MALDI: C₆₀H₁₁₃N₄O₂₆P₄S [M−H]⁻ calcd: 1461.60388, found: 1461.60491.

NBD Derivative 12b. Reaction of 11 (9.9 mg, 7.9 mmol) with NBD-X, SE (4.0 mg, 10.2 mmol) afforded 7.7 mg (5 mmol, 64%) of 12b. ¹H NMR (400 MHz, D₂O): δ 8.10-8.30 (m, 1H), 6.00-6.20 (m, 1H), 5.10-5.30 (m, 2H), 3.65-4.20 (m, 12H), 3.20-3.65 (m, 5H), 2.95-3.25 (m, 4H), 2.20-2.40 (m, 2H), 2.15-2.25 (m, 4H), 1.65-1.80 (m, 2H), 1.50-1.65 (m, 2H), 1.30-1.50 (m, 4H), 0.80-1.30 (m, 50H), 0.50-0.80 (m, 6H); ³¹P NMR (162 MHz, D₂O): δ 4.03, 2.92, 1.37, 0.47 (ratio 1:1:1:1). MS MALDI (free acid): 1161 (M−C₁₅H₃₁CO).

Fluorescein derivative 12c. Reaction of 11 (9.5 mg, 7.6 mmol) with 6-FAM-SE (4.7 mg, 9.8 mmol) yielded 9.1 mg (5.6 mmol, 74%) of 12c. ¹H NMR (400 MHz, D₂O): δ 7.30-7.50 (m, 8H), 5.20-5.40 (m, 2H), 3.10-4.30 (m, 14H), 2.20-2.40 (m, 4H), 1.40-1.60 (m, 4H), 0.90-1.40 (m, 48H), 0.60-0.90 (m, 6H); ³¹P NMR (162 MHz, D₂O): δ 4.94, 4.33, 1.21, 0.65 (ratio 1:1:1:1). MS MALDI (free acid): 1243 (M−C₁₅H₃₁CO), 1123 (M-fluorescein), 884 (M−C₁₅H₃,CO-fluorescein), 841 (M−C₁₅H₃₁CO-fluorescein-aminoethyl).

PROXYL derivative 12d. Reaction of 11 (9.8 mg, 7.8 mmol) with PROXYL-SE (2.9 mg, 10.1 mmol) afforded 6.1 mg (4.3 mmol, 55%) of 12d. MS MALDI (free acid): 1292 (M), 1123 (M-proxyl), 1053 (M−C₁₅H₃₁CO), 884 (M−C₁₅H₃₁CO-proxyl). HR MALDI: C₅₃H₁₀₃N₂O₂₅P₄ [M]⁻ calcd: 1291.57950; found: 1291.57679.

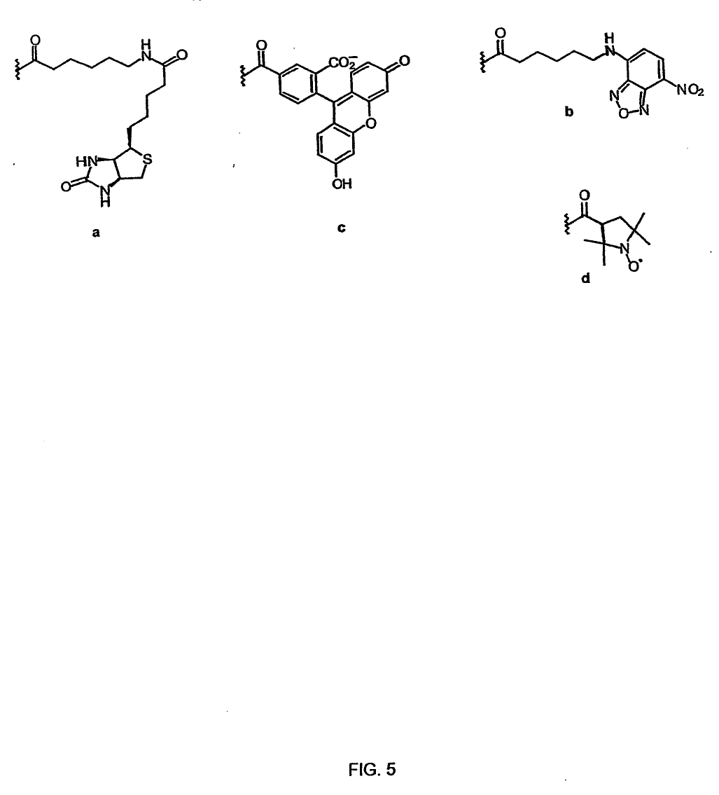

The resulting exemplary Pea-PIPₙ, 2,3-diacylthreitol-based Pea-Ptdlns(4,5)P₂, (“Pea-PIP2”) possesses a phosphatidylethanolamine (Pea) head group at the 1-position and a phosphatidylinositol 4,5-bisphosphate (Ptdlns(4,5)P₂) head group at the 4-position. Reporters (biotin, fluorophores, spin label) were covalently attached to the free amino group of the Pea, such that these reporters were targeted to the lipid-water interface. See FIG. 5. The diacyl moieties allow incorporation of Pea-PIP₂ into a lipid bilayer, while the Ptdlns(4,5)P₂ moiety in the aqueous layer is specifically recognized by Ptdlns(4,5)P₂-specific binding proteins. Reaction of the free Pea amino group of with four N-hydroxysuccinimidyl (NHS) esters afforded the corresponding biotinylated derivative C-2a, the fluorescent N-(7-nitrobenz-2-oxa-1,3-diazol-4-yl) (NBD) and 6-carboxyfluorescein derivatives C-2b and C-2c, and the spin-labeled 3-carboxy-2,2,5,5-tetramethyl-1-pyrrolidinyloxy (PROXYL) derivative C-2d. Preliminary biological results are described below for exemplary biotinylated and fluorescent derivatives.

### Example 2

### Synthesis of Pea-PIPₙs Having Eight Different Naturally Occurring Phosphoinositide Head Groups

Synthetic representatives of eight naturally occurring phosphoinositide head groups have been incorporated into Pea-PIPₙs of the present invention. The synthetic method for producing these was as described above for Pea-PI(4,5)P₂ and its reporter derivatives. The synthetic strategy for producing all eight of these Pea-PIPₙs is described in Table 1 and FIG. 2. The head groups used in this Example to produce Pea-PIPₙs of the invention are, Pi, PI(3)p, PI(4)P, PI(5)P, PI(3,4)P₂, PI(3,5)P₂, PI(4,5)P₂, PI(3,4,5)P₃.

TABLE 1

| Headgroup | Protected | Deprotected |
|---|---|---|
| PI, | R₁, R₁, R₃═Bn | R⁴, R⁵, R⁶═H |
| PI(3)p | R₁, R₂═Bn, R₃═PO₃Bn₂ | R¹, R²═H, R³═PO₃H₂ |
| PI(4)P | R₁, R₃═Bn, R₂═PO₃Bn₂ | R¹, R³═H, R²═PO₃H₂ |
| PI(5)P | R₂, R₃═Bn, R₁═PO₃Bn₂ | R², R³═H, R¹═PO₃H₂ |
| PI(3,4)P₂ | R₁═Bn, R₂, R₃═PO₃Bn₂ | R¹═H, R³═PO₃H₂ |
| PI(3,5)P₂ | R₂═Bn, R₁, R₃═PO₃Bn₂ | R²═H, R¹, R³═PO₃H₂ |
| PI(4,5)P₂ | R₃═Bn, R₁, R₂═PO₃Bn₂ | R³═H, R¹, R²═PO₃H₂ |
| PI(3,4,5)P₃ | R₁, R₂, R₃═PO₃Bn₂ | R¹, R², R³═PO₃H₂ |

### Example 3

### Synthesis of Linker-Modified Derivatives of Pea-PIPₙs

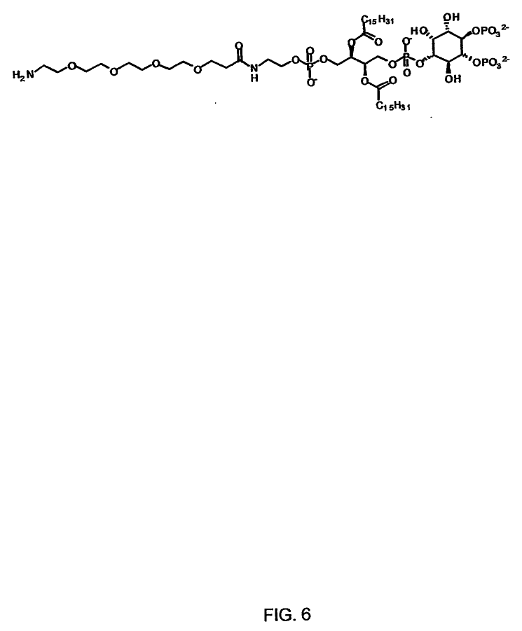

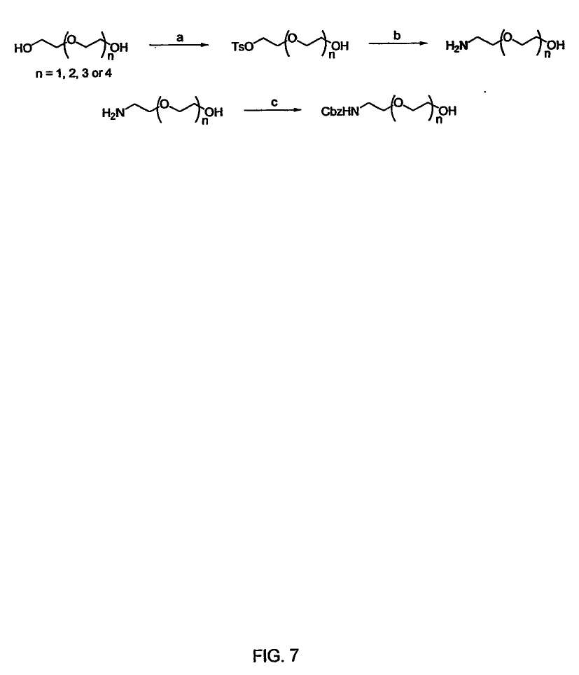

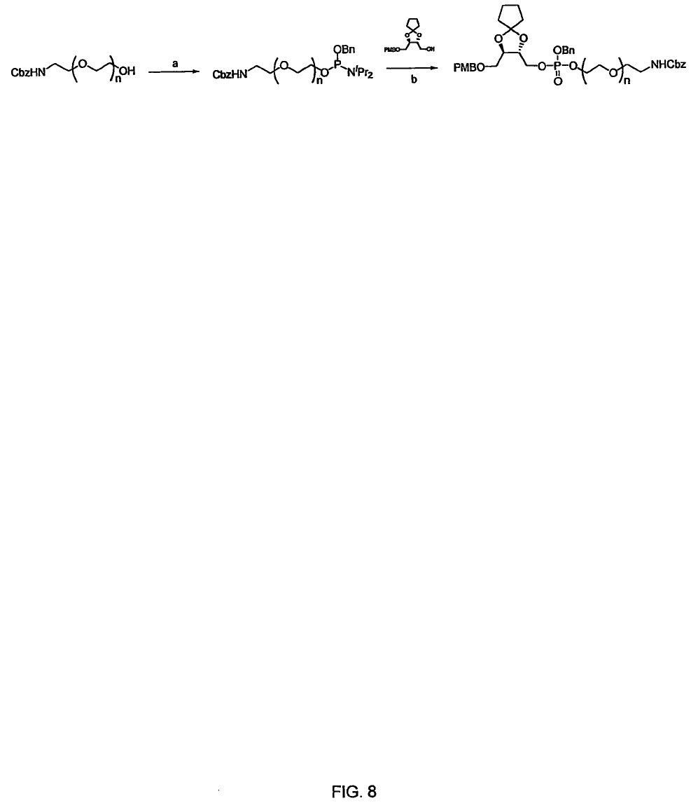

a. Hydrophilic linker-modified Pea-PIPₙ analogs. Preliminary data from immobilization of Pea-PIPₙs and a hydrophilic linker-modified analog to functionalized surfaces suggests that increasing the distance between the PIPₙ head group and the probe moiety may increase ligand recognition. A hydrophilic linker-modified Pea-PIPₙ derivative was synthesized in order to increase the distance between the PIPₙ head group and the probe moiety (see FIG. 6). In a first example of linker extension, amino-PEG-amide linker-extended Pea-PI(4,5)P₂ was prepared from the parent Pea-PI(4,5)P₂ by coupling the primary amine with the NHS ester of a 16-atom linker purchased as a Fmoc protected activated ester (available commercially, for example from Quanta Biodesign, Inc.). This derivative was examined for binding to the PLC_{δ} PH domain using AlphaScreen® (available from PerkinEimer Life Sciences), PIP Arrays™ (available from Echelon Biosciences, Inc.), and immobilized on NHS-activated plates or beads. Increased binding to a pleckstrin homology domain was observed with the addition of the hydrophilic linker.
b. Additional PEG linker Pea-PIPₙ analogs. Various lengths of poly(ethylene glycol)-based linkers can be used for preparation of alternative linker-modified Pea-PIPₙs. In a particular embodiment, PEG linkers were prepared from commercially available di-, tri-, tetra-, and/or penta(ethylene glycols). In the first step, the linkers were transformed into mono p-toluenesulfonates (Bauer, H., Sber, F., Petry, C., Knorr, A., Stadler, C., and Staab, H. A. (2001) European Journal of Organic Chemistry, 3255-3278), then converted into the oligo-PEG-\overline{ω}-aminoalcohols (Nelissen, H. F. M., Venema, F., Uittenbogaard, R. M., Feiters, M. C., and Nolte, R. J. M. (1997) Journal of the Chemical Society, Perkin Transactions 2: Physical Organic Chemistry, 2045-2053; Bramson, H. N., Corona, J., Davis, S. T., Dickerson, S. H., Edelstein, M., Frye, S. V., Gampe, R. T., Jr., Harris, P. A, Hassell, A., Holmes, W. D., Hunter, R. N., Lackey, K. E., Lovejoy, B., Luzzio, M. J., Montana, V., Rocque, W. J., Rusnak, D., Shewchuk, L., Veal, J. M., Walker, D. H., and Kuyper, L. F. (2001) Journal of Medicinal Chemistry 44, 4339-4358), and finally protected with CbzCl in dichloromethane/triethylamine (Roy, B. C., and Mallik, S. (1999) Journal of Organic Chemistry 64, 2969-2974). In this Example, linker extensions including 2-(2-Amino-ethoxy)-ethanol and 2-(2-[2-(2-Amino-ethoxy)-ethoxy]-ethoxy)-ethanol were introduced at an early stage of Pea-PI(4,5)P₂ synthesis, specifically during the preparation of the phosphoramidites. FIG. 7 illustrates the synthetic strategy for preparation of four oligo-PEG linkers. Relatively short PEG linkers are chosen to avoid floppiness, foldback, and heterogeneity often experienced with MW 700 or 1500 or 3400 PEG derivatives. In certain embodiments of the present invention, PEG linkers can be of any size. In particular embodiments, such PEG linkers may range from MW 88-3400 (44-20,000 Daltons). Suitably protected aminoalcohols are next converted into the corresponding phosphoramidites and coupled with specifically protected threitol as shown in FIG. 8. Following coupling, oxidation, and deprotection as previously described and shown in FIG. 2 and Table 1, three oligoethylene glycol phosphoramidite modified Pea-PIP, analogs were obtained.

### Example 4

### Pea-PIPₙs are Substrates for Lipid Phosphatase or Kinase

This Example demonstrates further that Pea-PIPₙs of the present invention are effective substrates for lipid phosphatases and kinases. In this Example, Pea-PIPₙs-were shown to be effective and specific substrates for recombinantly expressed and purified recombinant human PTEN/MMAC1 (acronyms for Phosphatase and Tensin homolog and mutated in multiple advanced cancers). PTEN/MMAC1 is a lipid phosphatase that removes the 3′ phosphate from PI(3,4,5)P₃ to produce PI(4,5)P₂. According to this Example, purified Glutathione S-Transferase (GST)-tagged, PTEN (0.24 mg/ml in 50% glycerol 50% elution buffer) was added to 125 pmol Pea-PI(3,4,5)P₃ in 50 ul reaction buffer (100 mM TRIS, pH 8.0, 10 mM DTT) (chemicals from Sigma-Aldrich Life Science, St. Louis, Mo.). The reactions were incubated for one hour at 37° C., and detected using an AlphaScreen™ assay. A standard curve of the product was generated by adding serial dilutions of Pea-PI(4,5)P₂ as the competitor to separate wells in the same plate. Results of this assay showed that two separate enzyme preparations of PTEN were active against Pea-PI(3,4,5)P₃. Using the standard curve, it was estimated that 48% of the Pea-PI(3,4,5)P₃ substrate was converted to Pea-PI(4,5)P₂ during the course of the reaction. These results show that members of this unique and novel class of phosphoinositide analogs are acted on as substrates in assay platforms for lipid kinases and phosphatases. Although this Example uses PTEN and detection of Pea-PI(4,5)P2; however, it is envisioned that this system extends to any combination of Pea-PIPn headgroup detection and any phosphoinositide kinase or phosphatase.

### Example 5

### Immobilized Pea-PIPₙs are substrates for Lipid Recognizing Proteins

In this Example, immobilized Pea-PIPₙs are readily and specifically recognized by lipid recognition proteins. According to this Example, six different quantities (from 200 pmol to 6 pmol) of Ptdlns(3,4)P₂, Ptdlns(3,5)P₂, Ptdlns(4,5)P₂, Pea-PI(4,5)P₂,Ptdlns(3,4,5)P₃; Pea-PI(3,4,5P)₃ PI and PE were spotted onto nitrocellulose, and binding of Glutathione S-Transferase (GST)-PLC δ₁-PH and GST-General Receptor for Phosphoinositides (Grp1)-PH constructs was examined by a protein-overlay technique (described in Dowler, S., Kular, G., and Alessi, D. R. (2002) Sci STKE 2002, PL6) (recombinant GST tagged PLC δ₁-PH and Grp1-PH domain proteins used in this Example were expressed in E. coli then purified using glutathione affinity resin (Amersham Biosciences, Piscataway, N.J.). The results of this binding assay show that neither lipid-recognizing protein (LRP) recognizes the PE or PI control lipids, nor do they recognize the non-cognate phosphoinositides, but both LRPs showed dose-dependent recognition of the correct immobilized phosphoinositide. Significantly, both Pea-PI(4,5)P₂ and Pea-PI(3,4,5)P₃ are able to bind the correct protein at lower concentrations than the corresponding diC₁₆ phosphoinositides, demonstrating that the Pea-PIPₙs have improved LRP binding capabilities compared to the current standard PIPₙ lipids.

### Example 6

### Biotinylated Pea-PIPₙs are Substrates for Lipid Recognizing Proteins

This example demonstrates that a phosphoinositide head-group specific lipid-recognizing protein (LRP), namely GST-Phospholipase Cδ₁ (PLC δ₁) PH domain, will easily bind to the biotinylated derivative of Pea-PI(4,5)P₂, even when bound to a surface.

According to this example, biotinylated lipid Pea-PI(4,5)P₂ was bound to a streptavidin-coated donor bead and the GST-tagged PLC δ₁—PH domain was attached to an anti-GST-coated acceptor bead. A luminescent signal quantitatively reported the interaction between the biotinylated lipid and the binding protein. Further, in the absence of a lipid or a specific binding protein, no signal was seen. In the presence of 0.1 pmol/well of specific binding protein, biotinylated Pea-PI(4,5)P₂ showed a dose-dependent increase in luminescent signal up to 1 pmol per well.

In this experiment, a bioluminescence assay (AlphaScreen™, Perkin-Elmer Life Sciences, Boston, Mass.) was used to establish biochemical relevance of the claimed compounds. Binding of Pea-PI(4,5)P2 to PLC δ₁ was determined using recombinant GST tagged PLC 5-PH domain protein that was expressed in E. coli, then purified using glutathione affinity resin (Amersham Biosciences, Piscataway, N.J.). Several concentrations of purified protein and biotinylated-Pea-PIP2 (all phosphoinositides were from Echelon Biosciences Inc, SLC, UT) were combined in a white 384-well microplate (Optiplate™, Packard Bioscience, Meriden, Conn.). Streptavidin donor and Anti-GST acceptor beads (Perkin-Elmer life sciences) were then added in a light protected area, so that the final bead concentration is 5 μg/mL in 25 μL final reaction volume (all dilutions in AlphaScreen assay buffer, Tris-Buffered Saline pH 7.5, 0.1% Tween-20, 0.1% Bovine Serum Albumin). The plate was protected from light and incubated for 2 hours at room temperature before reading with the AlphaScreen mode of a Fusion instrument (Perkin-Elmer life sciences).

In addition, competitive binding assays were conducted in which the GST-PLC δ₁-PH protein was pre-incubated for 30 min with 10 nM to 10 fM of unlabeled diC₄ Ptdlns(4,5)P₂, Ptdlns(3,4,5)P₃, or Pea-PI(4,5)P₂ prior to addition of the other reagents, using biotinylated Pea-PI(4,5)P₂ as the probe lipid. Over 100-fold selectivity was observed for displacement of the GST-PLC δ₁-PH from binding to Pea-PI(4,5)P₂ by the di-C₄ Ptdlns(4,5)P₂, relative to di-C₄ Ptdlns(3,4,5)P₃. A further increase in binding to the PH domain of GST-PLC 61 was observed using the non-biotinylated version of Pea-PI(4,5)P₂.

### Example 7

### Specific Recognition and Binding by Anti-PIP, Antibody to Pea-PIPₙs Covalently Bound to a Surface

The ability of lipid recognition proteins to specifically recognize and bind to Pea-PIPₙs was tested by coupling Pea-PIPₙs (as well as amino PIPs with the same headgroups as controls) to polystyrene microtiter plates. Briefly, 50 pmol per well of amino PI(4,5)P₂ (PIP2), amino PI(3,4,5)P₃ (PIP3), Pea-PI(4,5)P₂ (Pea-PIP2), and Pea-PI(3,4,5)P₃ (Pea-PIP3) lipids in 100 μL PBS were coupled via their primary amine functional groups to triplicate wells of a Maleic-Anhydride activated 96-well plate (Pierce, Rockford, Ill.). Underivatized groups were reacted with 200 μL Tris-Glycine-SDS overnight before blocking with 200 μL 0.02% Ovalbumin in TBS. The plate was then incubated with anti-PIP₃ antibody (for example, NN111.1.1, MBL International, Watertown, Mass.) for one hour at room temperature on an orbital shaker. The plate was washed 3-5 times with 200 μL/well of TBS containing 0.1% Tween-20. Specific binding was subsequently visualized by incubating with anti-mouse-HRP (horseradish peroxidase) secondary antibody (Sigma, St Louis, Mo.), washing as before, then adding tetramethylbenzadine (TMB) developing reagent (Sigma) and reading the absorbance at 450 nm in a plate reader. Anti-PIP3 antibody correctly and specifically bound to Pea-PI(3,4,5)P₃, demonstrating that, similar to the control lipids, Pea-PIP₃ (but not Pea-PIP₂) was recognized by the specific antibody. In addition, Pea-PIP₃ gave an increased signal compared to ω-amino alkanoyl PIP₃. These results show that immobilizing Pea-PIPs either by non-specific adsorption or covalent coupling allows the structural features necessary for recognition by antibodies and lipid recognizing proteins (namely, the headgroup and two fatty-acyl chains) to be better positioned than in traditional PIPs.

### Example 8

### Intracellular uptake of Pea-PIPs

Pea-PIPₙs can be delivered into living cells using a commercially available system (Echelon Bioscience Shuttle PIP™ system (Salt Lake City, Utah)). A fluorescent Pea-PI(4,5)P₂ analog was delivered to cells using the Shuttle PIP™ technology. This method has previously been used to deliver fluorescent PtdlnsPₙ analogs, Ptdlns(3,4)P₂ for Protein Kinase B (Akt) activation, and Ptdlns(3,4,5)P₃ to induce cell migration. 3T3L1 preadipocyte cells were seeded onto an 8-well cover-glass chamber slide in complete media. After 24 hrs the cells were approximately 60% confluent and the media was replaced with 100 μL serum free media for 45 minutes before adding a mixture of fluorescent Pea-PI(4,5)P₂-NBD and Histone H1 carrier premixed and incubated at room temperature for 10 minutes, final concentration of Pea-PI(4,5)P₂-NBD was 12.5 μM; and Histone carrier, 2.5 μM). After 30 minutes, the cells were imaged with a Bio-Rad confocal microscope at 300× magnification. Results of this assay showed that Pea-PI(4,5)P₂-NBD localized to intracellular compartments with bright staining associated in specific regions of the plasma membrane. This pattern of intracellular localization positions Pea-PI(4,5)P₂ correctly in the cell to substitute for endogenous Ptdlns(4,5)P₂ in signaling pathways and cell-based assays.

### Example 9

### PeaPIPns can substitute for synthetic PIPns on PIP Strip* Products

Pea-PIPₙs can substitute for synthetic PIPₙs on PIP Strip* products (Echelon, Salt Lake City, Utah). Briefly, 100, 50, 25, 12.5, 6.25, and 3.125 pmol of lipids in organic solvent were spotted onto PVDF membrane (Amersham, Boston, Mass.) and allowed to dry before blocking the membrane with 0.1% Ovalbumin in TBS. The membranes were then incubated with GST-PH domain proteins (LRPs) specific for PI(4,5)P2 or PI(3,4,5)P₃ for one hour at room temperature on an orbital shaker. Binding was visualized by subsequent incubations of anti-GST and anti-Mouse-HRP secondary antibodies followed by ECL (enhanced chemiluminescence) detection and exposure to photographic film. The Pea-PIPn's demonstrated the correct specificity for LRP binding and were superior to regular synthetic PIPs by immobilizing both PI(4,5)P₂ and PI(3,4,5)P₃-specific proteins at lower lipid concentrations.

### Example 10

### Pea-PIPs are capable of Adhesion to Microtiter Plates

Binding specificity of Pea-PIPs was further tested by coupling Pea-PIP2 and Pea-PIP3 (as well as amino PIPs with the same headgroups as controls) to polystyrene microtiter plates. Briefly, 50 pmol per well of amino PI(4,5)P₂ (PIP₂), amino PI(3,4,5)P₃ (PIP₃), Pea-PI(4,5)P₂ (PEA-PIP2), and Pea-PI(3,4,5)P₃ (PEA-PIP₃) lipids in 100 μL PBS were coupled via their primary amine functional groups to triplicate wells of a Maleic-Anhydride activated 96-well plate (Pierce, Rockford, Ill.). Underivatized groups were reacted with 200 uL Tris-Glycine-SDS overnight before blocking with 200 uL 0.02% Ovalbumin in TBS. The plate was then incubated with anti-PIP₃ antibody (for example, NN111.1, MBL International, Watertown, Mass.) for one hour at room temperature on an orbital shaker. The plate was washed 3-5 times with 200 μL/well of TBS containing 0.1% Tween-20. Specific binding was subsequently visualized by incubating with anti-mouse-HRP secondary antibody (Sigma, St Louis, Mo.), washing as before, then adding Tetramethylbenzadine (TMB) developing reagent (Sigma) and reading the absorbance at 450 nm in a plate reader. Similar to the control lipids; Pea-PIP₃ (but not Pea-PIP₂) was recognized by the specific antibody. In addition Pea PIP₃ gave an increased signal compared to amino PIP₃, similar to the nitrocellulose experiment. This Example demonstrates that immobilization of Pea-PIPs either by non-specific adsorption or covalent coupling allows the structural features necessary for recognition by antibodies and lipid recognizing proteins (namely, the headgroup and two fatty-acyl chains) to be better positioned than traditional PIPs.

### Example 11

### Pea-PIPs are Capable of Transfer into Living Cells

Pea-PIPₙs can be delivered into living cells using Echelon's Shuttle PIP system (Salt Lake City, Utah). 3T3-L1 fibroblasts in modified DMEM media (Gibco BRL, Maryland) were seeded onto an 8-well coverglass chamber slide ((Nalge Nunc International, Naperville, Ill.), 200 μL per chamber in complete media. After 24 hrs the cells were 60% confluent and the media was replaced with 100 μL serum free media for 45 minutes before 2.5 μL of 5 mM fluorescent Pea-PI(4,5)P₂-NBD was added to 6.25 μL of 200 μM Histone H1 (Sigma, St. Louis, Mo.) and incubated at room temperature for 10 minutes. Then 16.25 μL serum-free media was added to the PIP/Histone complex and incubated for an additional 5 minutes before it was added to the cells in a final volume of 125 μL and a final concentration of 100 μM Pea-PI(4,5)P₂ and 10 μM Histone H1. The cells on coverslips were imaged with a Bio-Rad confocal microscope at 60× magnification. This procedure was repeated in a separate experiment with a final Pea-PI(4,5)P₂ concentration of 12.5 μM and Histone concentration of 2.5 μM then visualized at 300× magnification. Pea-PI(4,5)P₂-NBD clearly localized to intracellular compartments with bright staining associated in several regions of the plasma membrane. This pattern of intracellular localization positioned Pea-PI(4,5)P₂ correctly in the cell to substitute for endogenous PI(4,5)P₂ in signaling pathways and cell-based assays.

### Example 12

### Pea-PIPn's can substitute for synthetic PIPs in lipid kinase and lipid phosphatase assay development programs

A Phosphoinositide head-group specific lipid-recognizing protein (LRP), namely GST-Phospholipase Cd1 PH domain, will easily bind to the biotinylated derivative of Pea PI(4,5)P₂. An AlphaScreen technology (Perkin-Elmer Life Sciences, Boston, Mass.) is utilized for our lipid-protein binding assay in this example.

AlphaScreen (for Amplified Luminescent Proximity Homogenous Assay) is a chemiluminescent, bead-based assay performed in white micro-titer plates. When excited by 680 nm laser light, donor beads convert ambient oxygen to a more excited singlet state. When an acceptor bead is in close proximity to the donor bead (through a biological interaction) singlet oxygen reacts with a thioxene derivative in the acceptor bead generating chemiluminescence light of 370 nm wavelength which further excites flourophores on the same acceptor bead emitting light at 520-620 nm.

Binding of PEA PI(4,5)P₂ to PLCd1 was determined by expressing recombinant GST tagged PLC-d1-PH domain protein in E. coli which was purified using glutathione affinity resin (Amersham Biosciences, Piscataway, N.J.). Several concentrations of purified protein and biotinylated-PEA PIP₂ (all phosphoinositides were from Echelon Biosciences Inc, SLC, UT) were combined in a white 384-well microplate (Optiplate™, Packard Bioscience, Meriden, Conn.). Streptavidin donor and Anti-GST acceptor beads (Perkin-Elmer life sciences) were then added in a light protected area, so that the final bead concentration is 5 μg/mL in 25 μL final reaction volume (all dilutions in AlphaScreen assay buffer, Tris-Buffered Saline pH 7.5, 0.1% Tween-20, 0.1% Bovine Serum Albumin). The plate was protected from light and incubated for 2 hours at room temperature before reading with the AlphaScreen mode of a Fusion instrument (Perkin-Elmer life sciences).

A strong binding interaction between biotinylated Pea-PI(4,5)P₂ and the PH domain of PLC-δ-1 was observed with maximum signal at 0.8 pmol/well biotinylated Pea-PI(4,5)P₂ and 0.4 pmol/well PLC-δ-1. Half these concentrations also produced a strong signal and were used as binding partners in the next series of experiments to demonstrate specificity of Pea-PIP binding to PLC-δ 1. These competitive binding experiments were similar to the binding experiment described above except that non biotinylated lipids are added to each reaction to compete with biotinylated Pea-PI(4,5)P₂ for binding to PLC-δ 1-PH domain. For example, eight concentrations of PI(3,4,5)P₃ diC4, PI(4,5)P₂ diC4, and Pea-PI(4,5)P₂ were added as competitors to separate wells in addition to 0.2 pmol/well PLC-δ-1-PH domain, 0.4 pmol/well of Pea-PI(4,5)P₂, streptavidin donor, and anti-GST acceptor beads; and incubated as previously described. It was determined that Pea-PI(4,5)P₂ was the best competitor with an IC50 value of 140 nM, and the incorrect headgroup PI(3,4,5)P₃ demonstrated the least affinity with an IC50 value of 70 mM. This 500 times better affinity of Pea-PI(4,5)P₂ for PLC d1-PH domain compared to PI(3,4,5)P₃ is fundamental to the development of a robust assay where one detects the specific PIPn product of a kinase or phosphatase in the presence of excess PIPn substrate.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A functionalized phosphoinositide polyphosphate comprising a carbon backbone, a phosphatidylethanolamine head group at carbon position 1, and a PtdlnsPₙ head group at carbon position 4.

2. A functionalized phosphoinositide polyphosphate of claim 1, wherein said carbon backbone is selected from the group consisting of 2,3-diacylthreitol, erythritol and a synthetic module.

3. A functionalized phosphoinositide polyphosphate of claim 2, wherein said synthetic module is prepared and coupled with a PtdlnsPₙ head group.

4. A functionalized phosphoinositide polyphosphate of claim 3, wherein said PtdlnsPₙ head group is selected from the group consisting of (PI, PI(3)ₚ, PI(4)P, PI(5)P, PI(4,5)P₂, PI(3,4)P₂, PI(3,5)P₂, PI(3,4,5)P₃).

5. A functionalized phosphoinositide polyphosphate of claim 1, further comprising a reporter group.

6. A functionalized phosphoinositide polyphosphate of claim 5, wherein the reporter is selected from the group consisting of a flourophore, a spin label, biotin, a radio label, a chemiluminescent label, a photophore, a chromophore, a nanogold particle and mixtures thereof.

7. A functonalized phosphoinositide polyphosphate of claim 6, wherein the flourophore is selected from the group consisting of acrylodan, AMCA, BODIPY, Cascade-Blue, CINERF, dansyl, dialkylaminocoumarin, eosin, erythrosine, fluorescein, hydroxycoumarin, NBD, Oregon green, PyMPO, pyrene, rhodamine, Rhodol Green, TMR, Texas Red, and X-Rhodamine.

8. A functionalized phosphoinositide polyphosphate of claim 5, wherein the reporter is covalently attached to a free amino group on the phosphatidylethanolamine head group.

9. A functionalized phosphoinositide polyphosphate of claim 8, wherein the reporter is attached to a free amino group by a linker.

10. A functionalized phosphoinositide polyphosphate of claim 9, wherein said linker comprises an oligo-polyethylene glycol linker.

11. A functonalized phosphoinositide polyphosphate of claim 9, wherein said linker may include an additional or alternative linker.

12. A functionalized phosphoinositide polyphosphate of claim 11, wherein said additional or alternative linker is selected from the group consisting of a diamino linker, a linker utilizing both phospho- and non-phospho linked spacers, a phosphatidylserine linker and a carboxylic acid linker.

13. A functionalized phosphoinositide polyphosphate of claim 11, wherein said linker includes aminoalcohols, heteroatom-containing derivatives or branched aminoalcohols.

14. A functionalized phosphoinositide polyphosphate of claim 13, further comprising a diamino linker.

15. A functionalized phosphoinositide polyphosphate of claim 1 selected from the group consisting of Pea-PI, Pea-PI(3)P, Pea-PI(4)P, Pea-PI(5)P, Pea-PI(3,4)P₂, Pea-PI(3,5)P₂, Pea-PI(4,5)P₂ and Pea-PI(3, 4,5)P₃.

16. A functionalized phosphoinositide polyphosphate of claim 1, wherein the carbon backbone comprises 2 acyl chains having from 2 to 26 carbons.

17. A functionalized phosphoinositide polyphosphate of claim 1, wherein the carbon backbone comprises an ether chain in the place of one or both acyl chains.

18. A functionalized phosphoinositide polyphosphate of claim 1, further comprising one or more phosphate groups that have been chemically modified to stabilize the compound against chemical or enzymatic hydrolysis.

19. A functionalized phosphoinositide polyphosphate of claim 1, further comprising a polymerizable group.

20. A method of screening for phosphoinositide-specific binding proteins comprising (a) contacting a functionalized phosphoinositide polyphosphate comprising a carbon backbone, a phosphatidylethanolamine head group at carbon position 1, and a PtdlnsPₙ head group at carbon position 4 with a putative phosphoinositide-specific binding protein-containing composition; and (b) measuring binding

21. A method of claim 20, wherein said functionalized phosphoinositide polyphosphate is attached to a surface.

22. A method of claim 21, wherein said surface is selected from the group consisting of plates, beads, liposomes, nitrocellulose and metals.

23. A method of claim 21, wherein said attaching a functionalized phosphoinositide polyphosphate is selected from the group consisting of streptavidin and NHS activation.

24. A method of claim 20, wherein said functionalized phosphoinositide polyphosphate is selected from the group consisting of Pea-PI, Pea-PI(3)P, Pea-PI(4)P, Pea-PI(5)P, Pea-PI(3,4)P₂, Pea-PI(3,5)P₂, Pea-PI(4,5)P₂ and Pea-PI(3, 4,5)P₃.

25. A method of claim 20, wherein said functionalized phosphoinositide polyphosphate further comprises a reporter.

26. A method of claim 25, wherein said reporter is selected from the group consisting of a flourophore, a spin label, biotin, a radio label, a chemiluminescent label, a photophore, a chromophore, a nanogold particle and mixtures thereof.

27. A method of claim 20, wherein said method comprises an assay selected from the group consisting of an in vitro enzyme assay, an in vitro agonist assay, an in vitro antagonist assay, a cell-based assay, a lipid kinase activity assay, a protein kinase activity assay, a lipid phosphatase activity assay, a protein phosphatase activity assay, a phospholipase assay and a phosphatase activity assay.

28. A method of claim 20, wherein said phosphoinositide-specific binding protein is selected from the group consisting of protein kinases, phosphoinositide kinases, phosphoinositide binding proteins, inositol phosphate binding proteins, lipid phosphatases and phospholipases.

29. A method of identifying compositions that affect phosphoinositide recognition or signaling at a cell membrane comprising (a) contacting a functionalized phosphoinositide polyphosphate comprising a carbon backbone, a phosphatidylethanolamine head group at carbon position 1, and a PtdlnsPₙ head group at carbon position 4 with a composition that putatively affects phosphoinositide recognition or signaling; and (b) measuring recognition and signaling.

30. A method of claim 29, wherein said composition that putatively affects phosphoinositide recognition or signaling comprises a compound selected from the group consisting of a chemical, a natural product, and a synthetic compound.

31. A method of claim 29, wherein said composition that putatively affects phosphoinositide recognition or signaling comprises a compound selected from the group consisting of lipid phosphatases and phospholipases.

32. A method of claim 28, wherein said method comprises a cell-based assay.

33. A functionalized phosphoinositide polyphosphate comprising Pea-PI, Pea-PI(3)P, Pea-PI (4)P, Pea-PI(5)P, Pea-PI (3, 4)P₂, Pea-PI (3,5)P₂, Pea-PI(4,5)P₂ or Pea-PI(3,4,5)P₃.
